(19) United States
(12) Patent Application Publication
(10) Pub. No.: US 2003/0104457 A1
(43) Pub. Date: Jun. 5, 2003

(54) Method and device for detecting and monitoring alcoholism and related diseases using microarrays

(75) Inventors: Adron Harris, Austin, TX (US); Dayne Mayfield, Austin, TX (US); Joanne Lewohl, Brisbane (AU); Peter Dodd, Brisbane (AU)

Correspondence Address: CHALKER FLORES, LLP, 12700 PARK CENTRAL, STE. 455, DALLAS, TX, 75251, US

(73) Assignee: Board of Regents, The University of Texas System, Austin, TX (US)

(21) Appl. No.: 10/291,247

(22) Filed: Nov. 7, 2002

Related U.S. Application Data
(60) Provisional application No. 60/338,270, filed on Nov. 8, 2001.

(51) Int. Cl. C12Q 1/68; G06F 19/00; G01N 33/48; G01N 33/50; C07H 21/04; C12M 1/34

(52) U.S. Cl. 435/6; 435/287.2; 702/20; 536/23.2

(57) ABSTRACT

A device and method for detecting, diagnosing, and or monitoring alcoholism and related disease states is disclosed. The device includes a substrate and one or more alcoholism-specific nucleic acids attached to the substrate. The substrate is contacted by a sample collected from a person with alcoholism or alcohol abuse or an alcohol related disease state, wherein contact occurs under pre-selected binding conditions that provides information that can be collected and recorded by a computer.

### BACKGROUND OF THE INVENTION

The United States Government may own certain rights in this invention under DOD Grant No.: 517-9-8444, and claims priority to U.S. application Ser. No. 60/338,270 filed Nov. 8, 2001.

### TECHNICAL FIELD OF THE INVENTION

The present invention relates in general to the method and device for detecting, monitoring or diagnosing alcoholism and related disease states, and more particularly, to the method and device for analyzing the progression of alcoholism and related disease states in a subject, preferably a human patient, using microarrays.

Without limiting the scope of the invention, its background is described in connection with the method of detecting, diagnosing or monitoring alcoholism and its progression in a person using nucleic acid microarrays, as an example.

Heretofore, in this field, the diagnosis and or detection of alcoholism or alcohol abuse and related behaviors has in practice depended on interviews, review of past records, or clinical impression. (Allen, J P, Columbus M, and Fertig J. 1995. Assessment in Alcoholism Treatment: An Overview. In: NIAAA Treatment Handbook Series 4, Assessing Alcohol Problems: A Guide for Clinicians and Researchers. NIH Publication No. 95-3745. pp. 1-9) Generally, items from the medical history and clinical signs are combined to form a diagnostic index. In addition, these items can be compared with the alcohol consumption history, CAGE questionnaire, and early indicator questionnaires that also form part of the index.

One problem with this method is the difficulty in obtaining the index. For example the index is rarely based on data obtained from one questionnaire but instead requires that several be made in order to show reliability. This process means the questionnaire must be taken at a minimum of two different points in time and generally at least one week apart, which limits the immediacy of obtaining a diagnosis. Furthermore, questionnaires can be lengthy with no central or computerized means of scoring them. This is particularly inconvenient for measures such as the Alcohol Use Inventory and the Addiction Severity Index, which are not only lengthy but also include multiple scales. In addition, while procedures for administering many scales are straightforward, others, such as the Addiction Severity Index, the Comprehensive Drinker Profile, the Alcohol Timeline Followback procedure, and several diagnostic scales, require extensive training before they can be properly used. Few of the scales offer variable scoring based on gender or ethnicity, which limits their universal applicability.

To detect alcoholism and alcohol consumption in its early phase, the diagnostic values of a discriminant score and a combination of laboratory tests are often used. The discriminant scores, however, give low sensitivity and specificity and are often limited to the intake of alcohol. For example, discriminant scores formed from gamma-glutamyltransferase, acetaldehyde-induced hemoglobin fraction, and the mean corpuscular volume reveal a low sensitive and specificity of 72% and 73%, respectively, and the test is optimally performed on heavy drinkers. (Sillanaukee P. 1992. The diagnostic value of a discriminant score in the detection of alcohol abuse. Arch Pathol Lab Med 116:924-9) Using other test combinations may offer better results, but none work across all age, sex, or ethnic groups. This confirms the assumption that alcohol abuse in all phases, like alcoholism and heavy drinking, may not be detected optimally with the use of only one marker or even combinations of them.

Unfortunately, and in part due to the above limitations, there is no immediately available and standardized method for diagnosis or detection of alcoholism or alcohol abuse and its related behaviors. As a result, physicians frequently misdiagnose or underdiagnose alcohol-related disorders. In addition, when lacking a standardized method of diagnosis, it is difficult if not impossible to rapidly and routinely monitor disease progression because physicians cannot rely on information obtained solely from the patient. Alcoholics rarely disclose the true extent of their alcohol consumption, and often deny and minimize any association between their use of alcohol and their other symptoms or problems.

As evidenced by the foregoing explanation, there is a real need for a simple and reliable method to detect, diagnose, and monitor alcoholism or alcohol abuse. An early diagnosis of alcoholism or a related abuses would also allow constructive intervention therapy at a stage when predicted recovery from alcoholism or its related diseases would be more favorable. Moreover, the ability to accurately and rapidly monitor the progression of alcoholism and related disease states, including the pathologic effects of alcoholism, permits physicians to effectively treat patients early and allows researchers to better assess the efficacy of various treatment regimens for alcoholism and related diseases.

Thus, reliable, reproducible, and economic methods of detecting and monitoring progression of alcohol abuse and related disease are needed. Developing a systematic method and device that provides an accurate and objective, physiologic measure of alcohol consumption is a necessary measure for physicians in their decision-making process and for improving morbidity and mortality outcomes in these patients. The method and device would provide healthcare professionals and researchers with an unbiased and credible tool for monitoring patient compliance with treatment goals.

### SUMMARY OF THE INVENTION

Currently, the detection, diagnosis, and or monitoring of persons with alcoholism or alcohol abuse or related disorders is limited by the lack of a simple, rapid, and standardized method and device. Present methods, especially questionnaires and discriminant scores, also require significant time, cost and are often considered unreliable given their gender, age, and or ethnicity bias. In addition, alcoholics rarely disclose the true extent of their alcohol consumption, and often deny and minimize their symptoms or problems adding intrinsic unreliability to any technology that includes direct testing of the person. In addition, present measurements of alcohol consumption only measure immediate consumption and reveal nothing about the extent of the abuse or its progression.

In the area of alcoholism detection and management, a significant problem of current systems is that while there are several technologies that address alcoholic consumption, each react independently to alcohol intake. In addition, each technology is unique and often offers limited sensitivity, specificity and predictive value. These traditional technologies are often influenced besides by alcohol, by age, gender and various of substances and non-alcohol-associated diseases. For example, the Early Detection of Alcohol Consumption score, a linear discriminant function derived from the analysis of a combination of up to 35 blood chemistry and hematology analytes, performs best only when identifying heavy drinking in those age 40 and above. (Harasymiw J W, Bean P. 2001. Identification of heavy drinkers by using the early detection of alcohol consumption score. Alcohol Clin Exp Res 25:228-35) More importantly, present technologies that measure alcohol consumption only measure immediate consumption and reveal nothing about the extent of the abuse or its progression. Furthermore, they do not cover the entire time axis for alcohol consumption. Tests for biological markers require a specific time frame of detection. For example, one marker, ethyl glucuronide is thought to have a high sensitivity and specificity, but can only be detected for short time periods of up to 80 hours after alcohol is eliminated from the body. (Wurst F M, Kempter C, Metzger J, Seidl S, Alt A. 2000. Ethyl glucuronide: a marker of recent alcohol consumption with clinical and forensic implications. Alcohol 20:111-6)

Current alcoholism detection and management is based on principles that limit their broad usage due, in part, because they rely primarily on subjective information or on direct alcohol intake. It is recognized, as disclosed herein, that current detection and management technologies are afflicted by the same inefficiencies intrinsic to the use of the current technologies themselves. The present invention is based on the recognition that cost effectiveness, accuracy and reliability in current technologies is not available for detecting and diagnosing alcoholism and its related disorders.

More particularly, the present invention can be a device for detecting, diagnosing and monitoring alcoholism and related diseases comprising a solid support for sustaining a substrate and a substrate as a collection of one or more alcoholism-specific nucleic acids attached to the solid support. A substrate for use with the present invention is human nucleic acid target elements of peptide nucleic acids with different determinable sequences, such as genomic DNA, cDNA, oligonucleotides, RNA, single-stranded or double-stranded or any chemical modifications thereof. In an alternative embodiment the human nucleic acid target elements can be, e.g., alcohol-specific genes with sequences specific for structural, metabolic, transcriptional or other genes for cell signaling, immune response, and or cell-cell interactions that are expressed by alcoholics or alcohol abusers. The solid support sustaining the substrate is any microfabricated solid surface to which molecules may be attached through either covalent or non-covalent bonds. One advantage of using a microfabricated solid surface to which molecules may attach is that it promotes amino, carboxyl, thiol or hydroxyl molecular groups to be incorporated onto its solid surface, molecular groups that readily bind human nucleic acids.

The substrate sustained by the solid support can come in contact with a sample, e.g., a fluid collected from a person who is considered to be alcoholic, alcohol abusive or have an alcohol-related disease. In an alternative embodiment, the sample can be blood plasma, urine, semen, saliva, lymph fluid, meningeal fluid, amniotic fluid, glandular fluid, and cerebrospinal fluid, cells, or any other fluid, cell or body tissue preparation. The sample can be optionally fractionated to create a probe. Likewise, the sample or probe can be optionally tagged with a label that can be detected by an apparatus with a light source or a capacitor. For example, a probe that is tagged with a fluorescent label can be viewed by a light source, e.g., a fluorescent microscope. A programmed computer, as part of the apparatus, can be used to record information from the sample, e.g., the location and magnitude of the detectable change at each target element. Ratio information between the sample and a control can also be recorded. By control it is meant that a sample can be collected from a person who is not an alcoholic or alcohol abusive and processed in the same manner as the alcoholic sample. The recorded information from the sample and the control can be stored as raw and or ratio information, e.g., in a computer database and can be displayed as raw and or ratio information, e.g., from a programmed computer.

The present invention can also be a method for analyzing the progression of alcoholism and related disease states comprising the steps of contacting a sample obtained from a person considered to be alcoholic or alcohol abusive or has an alcohol-related disease with a microarray to allow binding and collecting information about the binding. The method of the present invention can further comprise the step of identifying detectable changes between the sample and the control, the detectable changes that can be recorded, processed, displayed and stored on a programmed computer. By using the method of the present invention as a series of analyses taken over time, the progression of the disease in an individual person can be observed. The present method can also be used to analyze the expression of alcohol-specific genes at a single point or over time with a microarray carrying sequences specific for structural, metabolic, transcriptional or other genes for cell signaling, immune response, and or cell-cell interactions that are expressed by alcoholics or alcohol abusers.

### BRIEF DESCRIPTION OF THE DRAWINGS

For more complete understanding of the features and advantages of the present invention, reference is now made to the detailed description of the invention along with the accompanying Figures.

FIG. 1 is a flow chart of the steps involved in the detection and monitoring of alcoholism or alcohol abuse device of the present invention; and

FIG. 2 is a graph with the results from blood total RNA after one day using the PAX gene and Ambion controls to evalute the level of expression detectable in blood from subjects for genes associated with alcoholism;

FIG. 3 is a graph comparing the levels of genes from different subjects at day 1 and day 3;

FIG. 4 is a reference graph that demonstrates control levels of expression on the gene arrays; and

FIG. 5 is a graph comparing reference to human blood sample gene expression detected from an individual blood sample.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiment discussed herein are merely illustrative of specific ways to make and use the invention and do not delimit the scope of the invention. Various modifications and combinations of the illustrative embodiments, as well as other embodiments of the invention, will be apparent to persons skilled in the art upon reference to the description. It is therefore intended that the appended claims encompass any such modifications or embodiments.

the present invention includes a device and method for detecting, diagnosing, and or monitoring alcoholism and related disease states. The device uses a substrate and one or more alcoholism-specific nucleic acids attached to the substrate, wherein substrate is contacted by a sample collected from a person with alcoholism or alcohol abuse or an alcohol related disease state, under pre-selected binding conditions that provides information that can be collected and recorded by a computer. The information can be compared to control information from a sample obtained from a person without alcoholism, alcohol abuse, or an alcohol related disease and yields gene expression information and diagnostic and or prognostic medical information about the person. The method includes the steps of contacting a sample obtained from a person considered to be alcoholic or alcohol abusive or to have an alcohol-related disease with a substrate to allow binding and collecting information about the binding. The information is recorded on a computer, compared to control information and yields gene expression information and diagnostic and or prognostic medical information about the person.

The following are terms as they apply to this application: Alcoholism is a continued, excessive or chronic use of alcohol, including alcoholism, alcohol abuse and any alcohol-related disease. A substrate is any microfabricated solid surface to which molecules may be attached through either covalent or non-covalent bonds. This includes, but is not limited to, Langmuir-Bodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is contemplated. This includes planar surfaces, and also spherical surfaces.

As used herein “one or more alcoholism-specific nucleic acid” and the like is either DNA, RNA, single-stranded or double-stranded and any chemical modifications thereof or protein nucleic acids. Modifications include, but are not limited to, those that provide other chemical groups that incorporate additional charge, polarizability, hydrogen bonding, electrostatic interaction, and fluxionality to the individual nucleic acid bases or to the nucleic acid as a whole. As used herein the term “pre-selected binding conditions” are those conditions that maximize the signal-to-noise ratio for the detection of one or more alcohol-specific nucleic acids, the products thereof, or the physiological result of a change in the expression of such a gene. An example of binding conditions is described herein below in conjunction is the use of a DNA microarray, as will be known to those of skill in the art of expression microarrays.

A “nucleic acid target element” is a determinable sequence that contains at least one peptide located at a different location on the substrate. The determinable sequence may include either DNA, RNA, single-stranded or double-stranded and any chemical modifications thereof. Modifications include, but are not limited to, those that provide other chemical groups that incorporate additional charge, polarizability, hydrogen bonding, electrostatic interaction, and fluxionality to the individual nucleic acid bases or to the nucleic acid as a whole. The determinable sequence can further be portions of structural, metabolic, transcriptional or other genes, including ones that code for a proteases, receptors, channels, synaptic proteins, cell-cell or cell-matrix interactions, immune or inflammatory responses, cell signaling, molecular chaperones or other carrier proteins, molecular synthesis, cell cycle regulation, cell growth, cell proliferation, or cell death.

A “sample” is any mixture of macromolecules obtained from a person, e.g., nucleic acids extracted from the tissue or cells from the source. Sample includes, but is not limited to, whole blood, portions of whole blood, blood plasma, urine, semen, saliva, lymph fluid, meningeal fluid, amniotic fluid, glandular fluid, and cerebrospinal fluid. This also includes isolated nucleic acids separated from all of the preceding. “Sample” also includes solutions or mixtures containing homogenized solid material, such as feces, cells, tissues, and biopsy samples. Samples herein include one or more that are obtained at any point in time, including diagnosis, prognosis, and periodic monitoring.

Alcoholism is a major health problem in Western countries, but there is little molecular information about the genetic changes associated with the disease or how these changes translate into the detection, diagnosis or monitoring of alcoholism or alcohol abuse. Alcohol affects all organs of the body; the primary target is the central nervous system, where it influences neurotransmission to produce intoxication. Long-term alcohol use can lead to addiction, dependence, or tolerance and the phenomena of tolerance and withdrawal have shaped hypotheses concerning the mechanism of drug dependence. Long-term drug abuse is likely to initiate an adaptive response at the cellular level: when the drug is removed the neuroadaptations are unmasked, leading to the manifestation of the withdrawal syndrome. Substantial evidence suggests that this adaptive process is mediated, at least in part, by changes in gene expression.

Sustained alcohol exposure results in changes in the expression of selected genes, including those coding for neurotransmitter receptors, hormones and their receptors, signaling molecules, molecular chaperones, transcription factors, and cytokines. (Miles 1995) Although most studies regarding alcoholism are carried out using animal models or by exposing cells in culture to ethanol, it has recently been reported that chronic alcoholism in humans results in changes in the expression of certain mitochondrial and GABA_{A} receptor subunit genes. (Fan L, van der Brug M, Chen W-B, Dodd P R, Matsumoto I, Niwa S, Wilce P A. 1999. Increased expression of a mitochondrial gene in human alcoholic brain revealed by differential display. Alcohol Clin Exp Res 23:408-413; Lewohl J M, Crane D I, Dodd P R. 1997. Expression of the alpha 1, alpha 2 and alpha 3 isoforms of the GABA_{A} receptor in human alcoholic brain. Brain Res 751:102-12)

It is not known whether alcohol directly effects alcohol-responsive genes, or acts through an indirect mechanism involving many systems. For example, changes in transcription factor activation or in second messenger systems may initiate gene expression cascades. Activation or repression of alcohol-responsive transcription factors is also likely to result in changes in the expression of those genes with the corresponding control elements (Miles 1995). Each of these possibilities would result in distinct patterns of gene expression. Such patterns are difficult to detect by traditional measurements of a few mRNAs, but are well suited to microarray analysis. (Iyer et al., 1999) Microarray analysis also provides a genome-wide, non-biased study of gene expression patterns. This could be of particular importance in studying a drug with potentially pleiotropic actions, such as alcohol. Furthermore, microarray analysis may be used to study the protein, neurotransmitters, cytokines or other molecules effected by alcohol and alcohol abuse.

Device for Detecting, Diagnosing or Monitoring Alcoholism and Related Disease States

The present invention creates a device for analyzing alcohol-specific gene expression thus overcoming the limitations that currently exist and limit the rapid and specific analysis of alcoholism or its potentially pathologic progression over time.

A microarray device may be used to yield gene expression information as well as diagnostic and or prognostic medical information about a person considered to be alcoholic, alcohol abusive, or who has an alcohol-related disease. The present invention demonstrates that it is possible to identify the presence, absence, or modifications in the expression of genes related to alcoholism in the blood of patients. By using blood as the source of the nucleic acids for detection, the present invention greatly simplifies and makes detection of patients or potential patients more amenable to widespread use. One or more alcoholism-specific nucleic acids attach to the surface of the substrate under conditions apparent to those of skill in the art of molecular biology.

The alcoholism-specific nucleic acids may be genomic DNA, cDNA, oligonucleotides, RNA, single-stranded or double-stranded and any chemical modifications thereof, including but not limited to chemical groups that incorporate additional charge, polarizability, hydrogen bonding, electrostatic interaction, and fluxionality to the individual nucleic acid bases or to the nucleic acid as a whole. Furthermore, the alcoholism-specific nucleic acids include human nucleic acid target elements of one or more peptide nucleic acid, each with different determinable sequences. Each peptide is at a different location on the substrate at a density of 100 to 10,000 target elements per square centimeter. The alcoholism-specific nucleic acids attached to the substrate may be similar, e.g., to commercially available microarrays such as the HuGeneFL chip from Affymetrix, Inc., that contains target elements interrogating approximately 5,600 full length human genes.

An important finding of the present invention is that the human nucleic acid target elements may be portions of alcohol-specific genes with sequences specific to structural, metabolic, transcriptional or other genes for cell signaling, immune response, and or cell-cell interactions that are specifically expressed by alcoholics, alcohol abusers, or those with an alcohol-related disease. As used herein, the term “alcoholism-specific nucleic acids” include those genes that show a statistically significant change in gene expression detectable by a nucleic acid microarray. The change in expression may be a statistically significant increase or decrease of gene expression, e.g., an increase or decrease (up-regulation or down-regulation), a complete lack of gene expression or the presence of expression of a gene not observed before. The change in expression may be of nucleic acids, of proteins or in the effect of the change in expression of a protein, e.g., enzymatic activity or the effects of the enzymatic activity (e.g., phosphorylation, post-translational modification to proteins, changes to carbohydrates, lipids, and the like).

Examples of “alcoholism-specific nucleic acids” detected using the present invention, include but are not limited to, at least a portion of one or more of the following genes: M6 neuronal glycoprotein, myelin associated glycoprotein, myelin-associated oligodendrocyte basic protein, myelin basic protein, myelin proteolipid protein, myelin-oligodendrocyte glycoprotein, myelin protein Po, oligodendrocyte-myelin glycoprotein, PMP2, PMP22, MAL gene, ApoD, ApoE, carbonic anhydrase II, 2′,3′-cyclic nucleotide 3′-phosphodiesterase, Galactocerebrosidase, Transaldolase, UDP-galactose ceramide galactosyltransferase, MyT1, Puralpha, Edg-2, glial fibrillary acidic protein, keratin 6B, beta m spectrin, protease, serine, 9 (neurosin), proprotein convertase subtilisin/kexin type 4, calpain, large polypeptide L3, protease, serine, 11 (IGF binding), transmembrane protease, serine 2, endothelin receptor type B-like (GPCR 37), aquaporin 1 (channel-forming integral protein), potassium inwardly-rectifying channel, subfamily J, member 10, glutamate receptor, AMPA 1, N-ethylmaleimide-sensitive factor, EGF-containing fibulin-like extracellular matrix protein 1, CD44 antigen, cadherin 18, tetraspan NET-6, interferon, gamma-inducible protein 16, major histocompatibility complex, class II, DR beta 1, small inducible cytokine subfamily C, member 1, epoxide hydrolase 1, microsomal (xenobiotic), proline dehydrogenase (proline oxidase), glutathione S-transferase M5, ubiquinol-cytochrome c reductase core protein II, serine/threonine kinase, TU3A protein (also known as dominant rapamycin resistance 1, DRR1), secreted frizzled-related protein 1, GTP binding protein, phospholipase A2 (14-3-3 protein), platelet-activating factor acetylhydrolase, isoform Ib, alpha subunit, regulator of G-protein signaling 4 (RGS-4), synuclein, alpha, ribophorin II, 130 kD Golgi-localized phosphoprotein, autoimmune regulator (automimmune polyendocrinopathy candidiasis ectodermal dystrophy), SRY-box 9 transcription factor, spliceosome-associated protein (U2 snRNP), retinoid X receptor, gamma, nuclear transcription factor, X-box binding 1, TATA box binding protein-associated factor, RNA polymerase II, F, basic transcription factor 3, macrophage stimulating 1 (hepatocyte growth factor-like), LIM domain only 2 (rhombotin-like 1), bone morphogenetic protein 7 (osteogenic protein 1), requiem, apoptosis response zinc finger gene, discoidin domain receptor family, member 1, CDC-like kinase 2, CDC-like kinase 1, tumor protein p53-binding protein, 2, Human growth/differentiation factor 1, RACH1 (complements rad 1-1 cell cycle checkpoint mutant), RAN (member RAS oncogene family), nel-like 2, sarcolipin, KIAA0043 gene product, ESTs, cysteine and glycine-rich protein 1, conserved gene amplified in osteosarcoma, KIAA0027 gene product, Homo sapiens clone 23916, KIAA0202 gene product, selenoprotein P, plasma, 1, chromosome 16 BAC clone CIT987SK-A-69G12, upregulated by 1,25-dihydroxyvitamin D-3, tight junction protein 2, HREV107-like protein, clone 23555 mRNA sequence, clone 25030 mRNA sequence, KIAA0237 gene product, KIAA0725 gene product, KIAA0293 gene product, neuroblastoma (nerve tissue) protein, reticulon 1, ESTs weakly similar to cAMP-regulated guanine nucleotide exchange factor II, ESTs weakly similar to gene pp21protein, or ESTs highly similar to KIAA0195, ubiquitin C, microtubule-associated protein 4, calcium dependent protease (small subunit, proteasome subunit z, gamma-aminobutyric acid (GABA) A receptor beta 2 subunit, glutamate/aspartate transporter II, lysosomal membrane glycoprotein-1 (LAMP 1), cardiac gap junction protein, autotaxin-t (atx-t), Ig superfamily cytotoxic T-lymphocyte-associated protein (CTLA-4), HLA-DR alpha heavy chain a class II antigen of the major histocompatibility complex (MHC), Human acyl-CoA thioester hydrolase, cytochrome c oxidase subunit Vic, vacuolar H+ ATPase E subunit, ATP synthase, lysozyme mRNA, prostaglandin D2 synthase, liver mRNA for glyceraldehyde-3-phosphate dehydrogenase, SURF-1, calmodulin, calcineurin A2, GDI-dissociation inhibitor RhoGDIgammma, 14.3.3 protein (a protein kinase regulator), hPTPA, protein kinase C zeta, small GTP-binding protein, S10, protein tyrosine phosphatase, testis-specific cAMP-dependent protein kinase catalytic subunit (C-beta isoform), ADP-ribosylation factor 3, 90 kD heat shock protein gene, heat shock protein HSPA2 gene, mitochondrial matrix protein P1 (nuclear encoded), histone H2A2, RNA polymerase II elongation factor SIII p 15 subunit, acidic ribosomal phosphoprotein P0, glycyl-tRNA synthetase, ribosomal protein L27a, pigment epithelium-derived factor, TRPM-2 protein, angiotensinogen, transferrin, melanoma ubiquitous mutated protein (MUM-1), KIAA0080 gene product, KIAA0084 gene product, and or KIAA0174 gene product, or combinations thereof.

Contact between the alcoholism-specific nucleic acids attached to the substrate and a sample occurs under selective binding conditions, apparent to those of skill in the art of molecular biology. Selective binding conditions are those that permit the highest signal-to-noise ratio, and may be achieved by modifying the buffer conditions (pH, salt concentrations, detergents, etc.), temperature, and the like. For example, the alcoholism-specific nucleic acids attached to the substrate and the sample can be exposed to reagents or chemicals that facilitate binding of the sample to the alcoholism-specific nucleic acids and then washed with additional reagents or chemicals that facilitate removal of unbound sample, thereby leaving only bound sample and without changing the molecular structure of the sample, alcoholism-specific nucleic acids, or the substrate. Alternatively, one or more samples may come in contact with the alcoholism-specific nucleic acids attached to the substrate, in which one or more samples may be tagged with a label. For example, the label can be a fluorescent or non fluorescent molecule that emits a signal upon exposure to light. The intended sample is one that is collected from an alcoholic hereto defined as a person considered to be alcoholic, alcohol abusive or to have an alcohol-related disease. The sample collected from the person can include but is not limited to any mixture of macromolecules such as blood plasma, urine, semen, saliva, lymph fluid, meningeal fluid, amniotic fluid, glandular fluid, and cerebrospinal fluid or body tissue preparation. One or more samples, that may have different or like origins, may be collected from the person for immediate or later use.

Binding of the sample with the alcoholism-specific nucleic acid attached to substrate provides various information that may be collected by a computer as a change in signal intensity. One advantage of the present invention is that binding may be detected by a light source, capacitor, ion or plasma beam, including light microscopy, radiography, chemiluminescence, fluorescence microscopy, confocal microscopy, interferometry, surface plasma resonance, mass spectroscopy, atomic force microscopy, scanning tunneling microscopy.

The computer can then be used to record the information, store the information in a database, and or display the information. In addition, the information that is collected can reveal the location and or magnitude of the detectable change in signal intensity at each human nucleic acid target element. The detectable change can be, e.g., a change in fluorescence, signal intensity, or a change in a physical parameter, such as electrical conductance or refractive index, at each target element. Furthermore, an information ratio can be determined between the sample information and information collected from a control, in which the control is from a sample obtained from a person not considered to be alcoholic, alcohol abusive or to have an alcohol-related disease. Control information is collected in parallel to information collected from the alcoholic. Notably, commercially available computer programs may be used to collect sample and control information. An advantage of the present invention is that the information that is collected may yield gene expression information and diagnostic and or prognostic medical information about the person.

Method for Detecting, Diagnosing or Monitoring Alcoholism and Related Disease States

The present invention may use commercially or non-commercially available microarrays in a method for detecting, monitoring and or diagnosing alcoholism or alcohol abuse. The present invention may also be a method for analyzing alcohol-specific gene expression or its potentially pathologic progression over time.

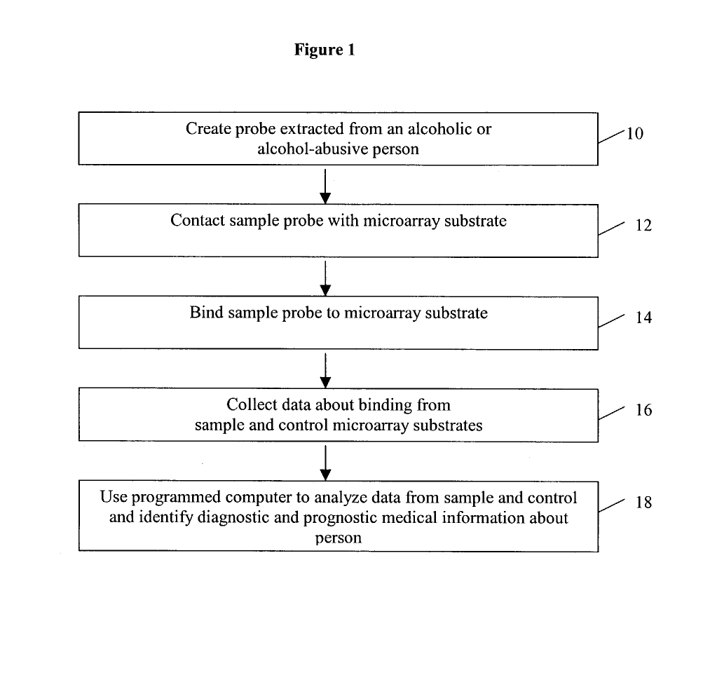

As shown in the flow chart of FIG. 1, the steps involved in a method that can yield diagnostic and prognostic medical information about a person. In step 10, a probe is created from a sample extracted from a person considered to be alcoholic, alcohol abusive or to have an alcohol-related disease. In step 12, there is contact between a substrate and a sample that, collected from an alcoholic hereto defined as a person considered to be alcoholic, alcohol abusive or to have an alcohol-related disease. The sample collected from the alcoholic can include but is not limited to any mixture of macromolecules such as blood plasma, urine, semen, saliva, lymph fluid, meningeal fluid, amniotic fluid, glandular fluid, and cerebrospinal fluid, cells, or any other fluid, cell or body tissue preparation. One or more samples with different or like origin, may be collected from the person for immediate or later use. One advantage of the present method is that one or more samples may be labeled with a fluorescent or non fluorescent molecule that emits a signal upon exposure to light as apparent to those of skill in the art of molecular biology. Alternatively, the sample can remain unlabeled and require a source other than fluorescent or non fluorescent light for its detection, e.g., source that records a physical parameter, such as electrical conductance or refractive index.

The substrate can be any microfabricated solid surface to which molecules may attach through either covalent or non-covalent bonds, such Langmuir-Bodgett films, glass, functionalized glass, germanium, silicon, PTFE, polystyrene, gallium arsenide, gold, silver, or any materials comprising amino, carboxyl, thiol or hydroxyl functional groups incorporated onto a surface. Another advantage of the present method is that the surface of the substrate can be planar or spherical. For example, the substrate can be a microarray that is commercially manufactured. Alternatively, the substrate may not be manufactured commercially. Attached to the substrate may be one or more human nucleic acid target elements. The attachment of the human nucleic acid target elements to the substrate occur under conditions apparent to those of skill in the art of molecular biology.

The human nucleic acid target elements may be genomic DNA, cDNA, oligonucleotides, RNA, single-stranded or double-stranded and any chemical modifications thereof, including but not limited to chemical groups that incorporate additional charge, polarizability, hydrogen bonding, electrostatic interaction, and fluxionality to the individual nucleic acid bases or to the nucleic acid as a whole. Furthermore, the human nucleic acid target elements may be one or more peptide nucleic acid, each with different determinable sequences. Each peptide is at a different location on the substrate at a density of 100 to 10,000 target elements per square centimeter. An important finding of the present invention is that the human nucleic acid target elements may be portions of genes with sequences specific to structural, metabolic, transcriptional or other genes, including ones that code for proteases, receptors, channels, synaptic proteins, cell-cell or cell-matrix interactions, immune or inflammatory responses, cell signaling, molecular chaperones or other carrier proteins, molecular synthesis, cell cycle regulation, cell growth, cell proliferation, or cell death.

The sample is allowed in step 12 to contact the microarray substrate, which can be either genomic DNA, cDNA, oligonucleotides, RNA, single-stranded or double-stranded and any chemical modifications thereof, including but not limited to chemical groups that incorporate additional charge, polarizability, hydrogen bonding, electrostatic interaction, and fluxionality to the individual nucleic acid bases or to the nucleic acid as a whole. The substrate is composed of human nucleic acids further comprises nucleic acid target elements of at least one peptide at a density of 100 to 10,000 target elements per square centimeter of surface area. One advantage of the present method is that one substrates may be used to contact one or more samples. Alternatively, the one or more substrates each comprised of the same or different human nucleic acid target elements may be used to contact one or more samples.

After contact between the sample and the substrate, the sample is allowed to bind to the substrate in step 14. Binding occurs under selective binding conditions apparent to those of skill in the art of molecular biology. For example, the sample and the substrate may be exposed to reagents or chemicals that facilitate binding of the sample to substrate followed by a wash with additional reagents or chemicals that facilitate removal of unbound sample, thereby leaving only bound sample without changing the molecular structure of the sample, or the substrate.

From the binding, information about the binding may be collected by a computer in step 16. The information may be collected as a change in signal intensity that can be detected by a light source, capacitor, ion or plasma beam, including light microscopy, radiography, chemiluminescence, fluorescence microscopy, confocal microscopy, interferometry, surface plasma resonance, mass spectroscopy, atomic force microscopy, scanning tunneling microscopy. The computer can record the information, store the information in a database, and or display the information. In addition, the information that is collected can reveal the location and or magnitude of the detectable change in signal intensity at each human nucleic acid target element. The detectable change may be, e.g., a change in fluorescence, signal intensity, or a change in a physical parameter, such as electrical conductance or refractive index, at each target element. Furthermore, an information ratio can be determined between the sample information and information collected from a control, in which the control is from a sample obtained from a person not considered to be alcoholic, alcohol abusive or to have an alcohol-related disease. Control information is collected in parallel to information collected from the alcoholic. Commercially available computer programs may be used to collect sample and control information. In step 18, the collected information can yield gene expression information and diagnostic and or prognostic medical information about the person.

Example of Detecting, Diagnosing or Monitoring Alcoholism and Related Disease States

Neuropathological studies show that chronic alcohol abuse results in brain shrinkage, particularly of the frontal lobes, which is largely due to a reduction in volume of the cerebral white matter (Reviewed in (Kril et al., 1997). Morphometric studies of gray matter indicate that neurons in specific regions of the brain, including frontal lobe cortical neurons, are selectively damaged (Kril J J, Harper C G (1989) Neuronal counts from four cortical regions of alcoholic brains. Acta Neuropathol (Berl) 79:200-4) In addition, the frontal cortex is susceptible to alcohol-induced damage and is important in judgement, decision making and other executive functions (Rahman S, Sahakian B J, Hodges J R, Rogers R D, Robbins T W (1999) Specific cognitive deficits in mild frontal variant frontotemporal dementia. Brain 122:1469-93; Godefroy O, Rousseaux M (1997) Novel decision making in patients with prefrontal or posterior brain damage. Neurology 49: 695-70) and explains why this brain regions is used for analysis by DNA microarrays.

A cDNA (UniGEMV) and an oligonucleotide (HuGeneFL) array are both used. Many genes are represented on these arrays, and there are also many genes unique to each array. Thus, use of both types of arrays allows cross-validation for some genes and study of a larger number of genes than would be possible with either array alone.

Sample Selection

Control and alcoholic cases are divided on the basis of alcohol intake. In accordance with National Health and Medical Research Council (NHMRC)/World Health Organization criteria, alcoholism is defined by an average daily intake of greater than 80 g of ethanol: many of the patients had consumed over 200 grams ethanol per day for most of their adult lives (usually >30 years). All controls are teetotalers or social drinkers who consume less (usually, much less) than 20 g of ethanol per day on average. Cases are matched for age at death, post-mortem delay, gender, cause of death, and drinking history where possible. Cases with a history of polydrug abuse were excluded. Samples may be taken by qualified pathologists under full ethical clearance #97/36 and informed written consent from the next of kin.

Two groups of control and two groups of alcoholic samples are selected; clinical details are given in Table 1. Each group (sample pool) includes five alcoholics and five matched control cases. The first set of cases are identical to those used in PCR-differential display experiments. The alcoholics in this group represent a heterogeneous population of three uncomplicated alcoholics, one alcoholic with cirrhosis and one alcoholic with concomitant Wernicke Encephalopathy. The selection criteria for Case Group Two is more stringent: only males are included, and any case with concomitant disease such as cirrhosis of the liver or Wernicke-Korsakoff Syndrome are excluded from the case set. In addition, care is taken to choose only those cases, controls or alcoholics, that exhibited no neuropathological abnormality at autopsy. Hence, the second case group is restricted to a subset of ‘uncomplicated’ alcoholics.

TABLE 1
Case Information

| | Age | PMD | Gender | Cause of Death | Neuropathology |
|---|---|---|---|---|---|
| Case Group One | | | | | |
| Controls | | | | | |
| 1 | 61 | 15 | M | coronary thrombosis | NSA |
| 2 | 54 | 16 | F | pulmonary embolism | NSA |
| 3 | 89 | 17 | M | cardiac failure | NSA |
| 4 | 73 | 22 | M | myocardial infarction | Alzheimer like changes |
| 5 | 70 | 46 | M | myocardial infarction | n/a |
| Alcoholics | | | | | |
| 1 | 69 | 30 | M | coronary atheroma | NSA |
| 2 | 70 | 16 | M | myocardial infarction | Wernicke-Korsakoff lesions |
| | | | | | (healed) |
| 3 | 43 | 21.5 | M | aspiration pneumonitis | NSA |
| | | | | coronary atherosclerosis | |
| 4 | 59 | 15 | M | suicide/hanging | NSA |
| 5 | 56 | 19 | M | cardiac failure | chronic hepatic |
| | | | | | encephalopathy |
| Case Group Two | | | | | |
| Controls | | | | | |
| 6 | 71 | 4.5 | M | acute renal failure | NSA |
| 7 | 75 | 36 | M | pneumonia | NSA |
| 8 | 67 | 67 | M | myocardial infarction | NSA |
| 9 | 52 | 61.4 | M | myocardial infarction | NSA |
| 10 | 67 | 34 | M | coronary atherosclerosis | n/a |
| Alcoholics | | | | | |
| 6 | 49 | 16 | M | cardiomyopathy | NSA |
| 7 | 34 | 31 | M | aspiration of gastric | n/a |
| | | | | contents | |
| 8 | 44 | 22 | M | cardiorespiratory arrest | NSA |
| 9 | 57 | 24 | M | drowning | NSA |
| 10 | 64 | 27 | M | ischemic heart disease | NSA |

Probe Preparation from the Sample

Total RNA was extracted using a modified guanidine isothiocyanate extraction procedure (Chomczynski and Sacchi 1987). Total RNA was extracted from each case individually and the samples pooled together to reduce (dilute) individual case-to-case differences that are unrelated to alcoholism. Each of the five cases making up each control and alcoholic group contributed 30 μg of total RNA to the pool. The same pooled control and alcoholic total RNA samples are used for hybridization to UniGEMV (Genome Systems Inc) and HuGeneFL (Affymetrix) arrays.

Analysis of Gene Expression Using cDNA Microarrays

PolyA⁺ RNA was extracted from 100 μg of total RNA using the Oligotex PolyA⁺ RNA Extraction kit from Qiagen (Valencia, Calif.). The protocol was carried out as per the manufacturer's instructions using a double-pass procedure. 200 ng of polyA⁺ RNA may be shipped to Genome Systems for probe generation and microarray hybridization.

Analysis of Gene Expression Using Oligonucleotide Arrays

For each sample, 25 μg of total RNA may be used as starting material for cDNA synthesis using a Superscript Choice kit (Gibco BRL Life Technologies, Rockville, Md.). In vitro transcription was then performed using a Bioarray RNA transcript labeling kit (Enzo, Farmingdale, N.Y.). The protocols are performed according to Affymetrix (Santa Clara, Calif.) recommendations. Prior to hybridization, biotin-labeled cRNA may be fragmented randomly to an average size of 30-60 bases by incubation at 94° C. for 35 min in 40 mM Tris-acetate pH 8.1, 100 mM potassium acetate and 30 mM magnesium acetate. Aliquots of fragmented cRNA (10 μg in a 200 μl master mix) are then hybridized to human oligonucleotide arrays (HuGeneFL, Affymetrix). The HuGeneFL chip contains probes interrogating approximately 5,600 full length human genes. The hybridization reactions may be conducted at 45° C. for 16 hr in a rotisserie oven (Affymetrix) set at 60 rpm. Hybridized arrays are then washed and stained with streptavidin-phycoerythrin (Molecular Probes) as described by Affymetrix protocols. Finally, arrays are scanned with a dedicated confocal microscope (Hewlett Packard, Santa Clara, Calif.).

Information Analysis

Analysis of cDNA microarray may be performed with a program called GEMTools (Incyte Pharmaceuticals). The program translates the 5600 target elements on the HuGeneFL chip into sequences for known genes. Genes can then be selected that pass a predefined default criteria for signal intensity above background and percent of a spot yielding signal in binding for both sample and control microarrays. Genes may be further filtered by selecting those that show a 1.4-fold increase or decrease in expression in the sample versus control.

Absolute and comparison analyses of oligo microarrays may be analyzed using GeneChip® Software 3.1 (Affymetrix). The total signal intensity of all oligo microarrays can be scaled to a uniform value by normalizing the average intensity of all genes (total intensity/number of genes) to a fixed value of 190. Under these conditions, the scaling factor for oligo microarrays may vary between 0.82 and 4.04. The protocols for analysis of Affymetrix arrays are described in detail (Lockhart et al., 1996; Wodicka et al., 1997), relevant portions incorporated herein by reference, as are the relevant portions of manuals provided by the manufacturer. The output of the GeneChip program includes data on signal intensity (“average difference”) and comparison between a sample and control (“fold-change”). Confidence measures for the presence or absence of a given mRNA probe (“absolute call”) and fold-change values (“difference call”) can be generated using a matrix-based decision algorithm. (Wodicka L, Dong H, Mittmann M, Ho M H, Lockhart D J. 1997. Genome-wide expression monitoring in Saccharomyces cerevisiae. Nat Biotechnol 15:1359-67) In all cases, the default values in the GeneChip program may be employed.

For oligonucleotide microarrays, genes with altered expression on may be selected by filtering data from comparison files generated between sample and control. The selection of genes represented by at least 10 target element pairs on the oligo microarrays, may include those with expression levels that differ from the control by at least about a 1.4 fold in both comparison files. Any “absent” gene as identified by the “absolute call” algorithms in the GeneChip program in any one of the bindings is eliminated from the candidate gene list.

Gene Expression Changes, Prognostic and Diagnostic Medical Information

Human alcoholics vary considerably in the mode, nature and duration of their alcohol abuse, especially with respect to the incidence of cycles of abuse and withdrawal. Alcoholics and non-alcoholics also exhibit considerable diversity in many pre-terminal factors, including environment, nutrition, medication, and genetic predisposition. To reduce the impact of these factors, one or more independent groups of controls may be used. In addition, one or more control group can be pooled for analysis (Table 1). Pooling of samples is used in recent microarray studies (Alizadeh et al., 2000) and reduces or dilutes the individual case-to-case differences. Furthermore, pooling of samples on the basis of similarity may delineate differences in gene expression from those due to population diversity. Samples from the same person may be pooled or run simultaneously to delineate small differences from artifact.

To identify genes that could underlie the adaptive response of brain regions particularly susceptible to alcoholic brain damage, comparisons may be made between controls and alcoholics using tissue from specific tissue, e.g. superior frontal cortex. (Kril J J, Harper C G. 1989. Neuronal counts from four cortical regions of alcoholic brains. Acta Neuropathol (Berl) 79:200-4). For example, pooled samples can be hybridized to cDNA and oligo microarrays and data can be expressed in terms of a differential expression ratio. The hybridizations performed using Case Group One pools are termed GS-1 (UniGEMV, Genome Systems) and A-1 (HuGeneFL, Affymetrix), and hybridizations for Case Group Two are termed GS-2 and A-2 (Table 1).

Microarrays are a relatively new technology which to date have been applied almost exclusively to cell culture systems and tumor samples, where differences in gene expression of two- to ten-fold are not uncommon. In animal models of alcohol abuse and in studies of human alcoholics conducted so far, the changes in gene expression which have been identified are relatively small: a 40% change in gene expression is common. Because of this, a relatively low threshold (1.4 fold) may be selected to identify genes of interest. In part because of the small changes expected in alcoholism, at least two different microarray substrates may be used. Furthermore, emphasis on genes with similar results in both case groups and both types of microarrays can be made.

The replicability of gene expression changes between controls and alcoholics in the two case groups is measured by the cDNA microarrays. The natural log of the differential expression ratios plotted for the two case groups shows consistent results, as reflected by the correlation coefficient (r) of 0.48. The replicability may also be judged by comparing how many genes show the same direction change between the two case groups at the chosen cutoff of 1.4-fold. For example, >100 genes show changes of at least 1.4-fold, up or down, in both case groups on the cDNA arrays. If this is due to random fluctuations, then an expectation of >100 genes may be used to show 1.4-fold changes of opposite direction between the two case groups. However, only 7 genes show an increase of at least 1.4-fold in one case group and a decrease of −1.4-fold in the second case group. Similarly, randomizing the data and then selecting for genes having at least 1.4-fold changes of the same direction in both case groups chooses far fewer genes than seen with the results shown herein. Thus, selecting genes beyond the 1.4-fold cutoff level in both case groups identifies a group of highly significant changes. While there may be some “false-positives,” it may be better to have a “relaxed” cutoff so as to avoid greater type II errors. Identifying a subset of functionally related genes within the selected list greatly increases the statistical power of the findings.

A striking finding from these studies is that most genes show similar expression in alcoholics and controls. The cDNA microarrays results in 3825 genes exceeding criteria as “present” while the oligo microarray analysis results in 705 genes being consistently called “present” in the four samples (2 control groups and 2 alcoholic groups). At least 64 genes may be found to increase and 99 decrease by 1.4-fold in both case groups as observed by either the cDNA or oligonucleotide microarrays. A ratio of 1.4 represents a 40% difference in expression level between the two samples on the microarray.

An initial inspection of the genes with altered expression indicates striking coordinate decreases in many mRNAs coding for myelin proteins. Both types of microarrays may be screened for genes related to myelin or oligodendrocytes. Over half (12/21) of these genes show decreases in expression (1.4-fold or greater) in at least two of the four hybridizations (Table 2).

TABLE 2
Myelin-related expression data

| CDNA | Oligo | | | | | |
|---|---|---|---|---|---|---|
| Accession | Accession | Gene Name | GS-1 | GS-2 | A-1 | A-2 |
| Structural | | | | | | |
| NR | U45955 | M6 neuronal glycoprotein | NR | NR | −1.6 | −1.5 |
| M29273 | M29273 | myelin associated glycoprotein | −2.1 | −1.9 | −1.7 | −1.7 |
| | | (MAG) | | | | |
| H23197 | D28114 | myelin-associated | −1.2 | −1.2 | −1.9 | 1.2 |
| | | oligodendrocyte basic protein | | | | |
| | | (MOBP) | | | | |
| NR | M13577 | myelin basic protein | NR | NR | −1.7 | −1.1 |
| M27110 | M54927 | myelin proteolipid protein (PLP) | −2.1 | −1.9 | −2.1 | −1.3 |
| NR | Z48051 | myelin-oligodendrocyte | NR | NR | −1.2 | −1.1 |
| | | glycoprotein (MOG) | | | | |
| D14720 | D10537 | Myelin protein Po | −1.2 | −1.1 | −1.2 | −1.7 |
| NR | L24893 | Myelin protein Po | NR | NR | 1.1 | 2.4 |
| M63623 | M63623 | oligodendrocyte-myelin | ND | ND | −2.7 | −1.2 |
| | | glycoprotein (OMGP) | | | | |
| D16181 | D16181 | PMP2 | −1.4 | −1.6 | −2.4 | −1 |
| AA777648 | U08049 | PMP22 | −1.5 | −1.8 | 1.4 | 1 |
| NR | U08096 | PMP22 | NR | NR | 3.3 | 1.3 |
| NR | D11428 | PMP22 | NR | NR | −5.7 | −4.8 |
| X76220 | X76223 | MAL gene | −2.3 | −2.3 | −2.6 | −3.5 |
| Metabolic | | | | | | |
| J02611 | J02611 | ApoD | −1.6 | −1.8 | −1.8 | −1.4 |
| AI188519 | NR | ApoD | −1.5 | −1.9 | NR | NR |
| K00396 | M12529 | ApoE | −1.3 | −1.6 | −1.3 | −1.6 |
| J03037 | Y00339 | Carbonic anhydrase II | −1.2 | −1.3 | −1.6 | −1.6 |
| D13146 | M19650 | 2′,3′-cyclic nucleotide | ND | ND | −1.7 | −2.5 |
| | | 3′-phosphodiesterase (CNP) | | | | |
| D13146 | D13146 | 2′,3′-cyclic nucleotide | ND | ND | −1.4 | 1 |
| | | 3′-phosphodiesterase (CNP) | | | | |
| L38559 | L23116 | Galactocerebrosidase (GALC) | ND | 1.4 | −1.2 | 1.4 |
| L19437 | L19437 | Transaldolase | −1.1 | −1.6 | −1.6 | 1.1 |
| AA811893 | U30930 | UDP-galactose ceramide | −1 | −1.4 | −1.9 | −1.7 |
| | | galactosyltransferase | | | | |
| | | (CGT, E.C. 2.4.1.45) | | | | |
| Transcription | | | | | | |
| AB020642 | M96980 | MyT1 | 1.1 | 1.2 | −1.2 | −1.8 |
| NR | X91648 | Puralpha | NR | NR | 1 | −1.9 |
| Other | | | | | | |
| AA059335 | U11861 | Edg-2 | NR | NR | −1.1 | 1.1 |

Myelin-associated glycoprotein (MAG), myelin and T cell differentiation protein (MAL), and apolipoprotein D (ApoD) show decreases in all four bindings. However, the changes in myelin gene expression may be selective because myelin-oligodendrocyte glycoprotein (MOG) and Edg-2, a tetraspan receptor linked with myelination, show no changes in expression and six other genes, including the abundant myelin basic protein, show decreased expression in only one binding. In contrast, galactocerebrosidase (GALC), the major enzyme important for degradation of galactosylceramide, is increased in expression in two out of four hybridizations. The observation that only a subset of myelin genes may decrease in expression argues against variation in tissue dissection (giving more white matter in controls) as a possible explanation.

The majority of the genes in Table 2 play a role in myelin structure (Boison and Stoffel 1994; Montag et al., 1994) but biosynthetic genes are also altered (Schaeren-Wiemers et al., 1995). Interestingly, this includes the genes for ApoD and ApoE that are both involved in lipid transport. These genes are also implicated in neuronal degenerative diseases such as Alzheimer's Disease (Roses 1997; Terrisse et al., 1998). It is also striking that the myelin-related transcription factors, MyT1 and Puralpha, are not consistently altered in the alcoholic samples, suggesting additional factors in the coordinate down-regulation of so many myelin genes.

Glial fibrillary acidic protein (GFAP) is a major structural protein of astrocytes that has been widely studied in animal models of alcohol abuse. GFAP is up-regulated after acute ethanol treatment but decreases with chronic treatment and may be transcriptionally regulated by ethanol (Valles et al., 1997). A deficiency in GFAP also results in demyelination (Liedtke et al., 1996): mice with a null mutation in the GFAP gene exhibit abnormal myelination and hydrocephalous associated with white matter loss. Hence, GFAP expression is required for the maintenance of myelinated fibers and white matter in the CNS.

The decrease in expression of myelin-related genes in the frontal cortex of alcoholics may indicate that chronic alcohol abuse either directly or indirectly affects the transcription of myelin-related genes, resulting in a decrease in the amount of myelin proteins. Alternatively, the loss of myelin gene expression may indicate that oligodendrocytes are particularly susceptible to the neurotoxic effects of ethanol.

Myelin gene expression is studied with less detail in animal models of alcohol abuse although alterations in myelin biogenesis are well-documented in animal models of Fetal Alcohol Syndrome (FAS). Rats prenatally exposed to ethanol exhibit delays in myelination (Jacobson S, Rich J, Tovsky N J (1979) Delayed myelination and lamination in the cerebral cortex of the albino rat as a result of the fetal alcohol syndrome. Currents in Alcoholism 5:123-133) which is likely to be the result of abnormalities in the temporal pattern of expression of myelin-related genes including GFAP, PLP, MAG, Mal and myelin basic protein (Naus and Bechberger 1991; Milner et al., 1987; Chiappelli et al., 1991). Exposure to ethanol postnatally (postnatal days 4-10) reduces the expression of specific myelin basic protein and MAG isoforms (Zoeller et al., 1994). White-matter loss and altered myelin biogenesis is documented in children with fetal alcohol syndrome (Riley et al., 1995)

Demyelination and white-matter loss is extensively documented in neuroimaging (Pfefferbaum et al., 1993) and neuropathological (Kril et al., 1997) studies of humans and are thought to account for the brain shrinkage seen in these cases. White-matter loss is most severe in cirrhotic alcoholics and alcoholics with concomitant WE and WKS but can also be found in uncomplicated alcoholics (Kril et al., 1997), suggesting that it results from a direct neurotoxic effect of alcohol. Chronic alcoholism results in atrophy of one of the main white matter structures in the brain, the corpus callosum. Corpus callosum size and thickness are significantly reduced in alcoholic males compared with age and sex matched controls (Oishi et al., 1999) as well as in children prenatally exposed to alcohol (Riley et al., 1995).

It is important to note that neuroimaging studies demonstrate that white matter loss is reversible with abstinence (Shear et al., 1994), and hence may represent a reversible change in myelination rather than an irreversible loss of axons or oligodendrocytes. Future microarray analyses of RNA extracted from the frontal cortex of abstinent alcoholics compared with non-drinking controls or cases known to be actively drinking at the time of death may reveal whether the changes in myelin gene expression are reversible. In addition, analysis of the expression of these genes in animal models of alcohol abuse may help define the period of abstinence required to reverse these changes.

Importantly, alcoholics also suffer a disproportionate incidence of central pontine myelinolysis and Marchiafava-Bignami disease (Miles and Diamond 1998). These demyelinating disorders may result from toxic or metabolic factors other than alcohol. Down-regulation of myelin gene expression may predispose alcoholics to myelin injury in central pontine myelinolysis and Marchiafava-Bignami disease.

Non-myelin genes can be arranged into functional groups using the criteria of differential expression ratios of 1.4 fold in both case groups with either cDNA arrays (Table 3) or oligonucleotide arrays (Table 4).

TABLE 3
Genes which meet the criteria for differential expression on the cDNA (GS-1,
GS-2) array: Data from oligonucleotide (A-1, A-2) arrays have been added
were possible

| | | | | | |
|---|---|---|---|---|---|
| Accession | GeneName | GS-1 | GS-2 | A-1 | A-2 |

Structural Proteins

| | | | | | |
|---|---|---|---|---|---|
| AA059335 | glial fibrillary acidic protein | −3.1 | −2.2 | −1.9 | −2.4 |
| L42611 | keratin 6B | −1.6 | −2.8 | N/D | −2.6 |
| AB008567 | beta III spectrin | 1.4 | 1.5 | N/R | N/R |

Proteases

| | | | | | |
|---|---|---|---|---|---|
| U62801 | protease, serine, 9 (neurosin) | −2.1 | −2 | −1.5 | −4 |
| D87993 | proprotein convertase subtilisin/kexin type 4 | −1.9 | −2.1 | N/R | N/R |
| AI978885 | calpain, large polypeptide L3 | −1.7 | −1.8 | N/R | N/R |
| Y07921 | protease, serine, 11 (IGF binding) | −1.5 | −1.6 | −1.9 | −1.6 |
| U75329 | transmembrane protease, serine 2 | 1.4 | 1.6 | N/D | N/D |

Receptors, Channels and other Synaptic Proteins

| | | | | | |
|---|---|---|---|---|---|
| Y12476 | endothelin receptor type B-like (GPCR 37) | −2.5 | −2.1 | −1.7 | −1.8 |
| U41517 | aquaporin 1 (channel-forming integral | −1.8 | −2.2 | −4.1 | −4.5 |
| | protein) | | | | |
| U52155 | potassium inwardly-rectifying channel, | −1.5 | −1.9 | N/D | N/D |
| | subfamily J, member 10 | | | | |
| M64752 | glutamate receptor, AMPA 1 | 1.4 | 1.6 | N/D | 2.2 |
| U03985 | N-ethylmaleimide-sensitive factor | 1.6 | 1.4 | N/R | N/R |

Cell-Cell or Cell-Matrix Interactions

| | | | | | |
|---|---|---|---|---|---|
| U03877 | EGF-containing fibulin-like extracellular | −1.6 | −2.9 | N/D | N/D |
| | matrix protein 1 | | | | |
| X55150 | CD44 antigen | −1.4 | −1.4 | N/D | N/D |
| U59325 | cadherin 18 | 1.4 | 1.7 | N/D | N/D |
| AF120265 | tetraspan NET-6 | 1.4 | 1.4 | N/R | N/R |

Immune or Inflammatory Response

| | | | | | |
|---|---|---|---|---|---|
| S75433 | interferon, gamma-inducible protein 16 | −1.6 | −1.4 | N/D | N/D |
| V00522 | major histocompatibility complex, class II, | −1.4 | −1.5 | N/R | N/R |
| | DR beta 1 | | | | |
| AL031736 | small inducible cytokine subfamily C, | 1.8 | 1.6 | N/D | N/D |
| | member 1 | | | | |

Metabolism

| | | | | | |
|---|---|---|---|---|---|
| J03518 | epoxide hydrolase 1, microsomal | −1.4 | −1.4 | −1.4 | 1.8 |
| | (xenobiotic) | | | | |
| U82381 | proline dehydrogenase (proline oxidase) | −1.4 | −1.9 | N/R | N/R |
| AI797367 | glutathione S-transferase M5 | −1.4 | −1.7 | N/D | N/D |
| J04973 | ubiquinol-cytochrome c reductase core | 1.5 | 1.4 | N/D | 1.7 |
| | protein II | | | | |

Cell Signalling

| | | | | | |
|---|---|---|---|---|---|
| AB004884 | serine/threonine kinase | −1.9 | −1.5 | N/D | N/D |
| AF089853 | TU3A protein (also known as dominant | −1.6 | −2.6 | 1.2 | −1.7 |
| | rapamycin resistance 1, DRR1) | | | | |
| AF056087 | secreted frizzled-related protein 1 | −1.5 | −1.4 | N/R | N/R |
| AF054183 | GTP binding protein | 1.4 | 1.4 | N/R | N/R |
| AL008725 | phospholipase A2 (14-3-3 protein) | 1.4 | 1.4 | 1.2 | 1.3 |
| L13385 | platelet-activating factor acetylhydrolase, | 1.4 | 1.5 | N/R | N/R |
| | isoform Ib, alpha subunit | | | | |
| AI651602 | regulator of G-protein signalling 4 (RGS-4) | 1.5 | 1.8 | N/D | 2.8 |
| L08850 | synuclein, alpha | 1.6 | 1.4 | −1.1 | 1.4 |

Transcription or Protein Synthesis

| | | | | | |
|---|---|---|---|---|---|
| Y00282 | ribophorin II | −1.8 | −1.8 | 1.1 | −1.1 |
| U55853 | 130 kD Golgi-localized phosphoprotein | −1.7 | −2 | −2.1 | N/D |
| AJ009610 | autoimmune regulator (automimmune | −1.5 | −1.6 | N/R | N/R |
| | polyendocrinopathy candidiasis ectodermal | | | | |
| | dystrophy) | | | | |
| S74506 | SRY-box 9 transcription factor | −1.4 | −1.9 | −1.5 | −1.5 |
| L35013 | spliceosome-associated protein (U2 snRNP) | −1.4 | −1.4 | N/R | N/R |
| U38480 | retinoid X receptor, gamma | −1.4 | −1.4 | N/D | N/D |
| U15306 | nuclear transcription factor, X-box binding 1 | 1.4 | 1.4 | N/D | N/D |
| X97999 | TATA box binding protein-associated factor, | 1.4 | 1.4 | 1.1 | 1.1 |
| | RNA polymerase II, F | | | | |
| Z48042 | basic transcription factor 3 | 1.4 | 1.4 | 1.1 | N/D |

Cell Cycle Regulation: Growth, Proliferation and Death

| | | | | | |
|---|---|---|---|---|---|
| U28054 | macrophage stimulating 1 (hepatocyte | −3 | −1.8 | N/D | N/D |
| | growth factor-like) | | | | |
| X61118 | LIM domain only 2 (rhombotin-like 1) | −2.3 | −1.8 | −1.7 | N/D |
| X51801 | bone morphogenetic protein 7 (osteogenic | −1.6 | −1.4 | N/D | N/D |
| | protein 1) | | | | |
| AF001433 | requiem, apoptosis response zinc finger gene | −1.5 | −1.5 | −1.3 | N/D |
| Z29093 | discoidin domain receptor family, member 1 | −1.5 | −2 | N/R | N/R |
| AF023268 | CDC-like kinase 2 | −1.5 | −1.5 | −1.1 | −1.2 |
| AA044407 | CDC-like kinase 1 | −1.5 | −1.6 | N/R | N/R |
| AI123916 | tumor protein p53-binding protein, 2 | −1.4 | −1.5 | −1.3 | −1 |
| AI936592 | Human growth/differentiation factor 1 | −1.4 | −1.5 | −1.7 | −1.7 |
| U35735 | RACH1 (complements rad1-1 cell cycle | −1.4 | −1.9 | 1 | −1.1 |
| | checkpoint mutant) | | | | |
| AF052578 | RAN, member RAS oncogene family | 1.4 | 1.4 | N/R | N/R |
| D83018 | nel-like 2 | 1.6 | 1.6 | −1.2 | 1.6 |

Other

| | | | | | |
|---|---|---|---|---|---|
| U96094 | sarcolipin | 1.7 | 1.7 | N/D | N/D |

Unknown

| | | | | | |
|---|---|---|---|---|---|
| AI888282 | ESTs | −4.2 | −2.9 | N/D | N/D |
| D26362 | KIAA0043 gene product | −2.4 | −2.2 | −1.6 | −1 |
| AI683914 | ESTs | −1.8 | −1.9 | N/R | N/R |
| M76378 | cysteine and glycine-rich protein 1 | −1.6 | −1.6 | −1.8 | −1.3 |
| AF022231 | conserved gene amplified in osteosarcoma | −1.6 | −1.5 | N/R | N/R |
| AA703765 | KIAA0027 gene product | −1.6 | −1.7 | −2.4 | −3.9 |
| F13434 | Homo sapiens clone 23916 | −1.6 | −2.6 | N/R | N/R |
| D86957 | KIAA0202 gene product | −1.5 | −1.4 | N/D | N/D |
| AI521556 | selenoprotein P, plasma, 1 | −1.5 | −1.7 | −1.2 | −1.7 |
| AI677769 | ESTs | −1.4 | −1.4 | N/R | N/R |
| AC002550 | chromosome 16 BAC clone CIT987SK-A- | −1.4 | −1.6 | N/R | N/R |
| | 69G12 | | | | |
| S73591 | upregulated by 1,25-dihydroxyvitamin D-3 | −1.4 | −1.4 | −2.3 | −4.2 |
| L27476 | tight junction protein 2 | −1.4 | −1.6 | N/R | N/R |
| AI375733 | HREV107-like protein | −1.4 | −1.6 | N/R | N/R |
| AI936592 | ESTs | −1.4 | −1.5 | −1.7 | −1.7 |
| AA235116 | ESTs | 1.4 | 2.4 | −1.5 | N/D |
| AA102395 | ESTs | 1.4 | 1.7 | N/D | N/D |
| AI003191 | ESTs | 1.4 | 1.4 | N/R | N/R |
| AI375991 | ESTs | 1.4 | 1.6 | N/R | N/R |
| AA283901 | ESTs | 1.4 | 1.5 | N/D | N/D |
| W05842 | ESTs | 1.4 | 2.1 | N/R | N/R |
| AI669775 | ESTs | 1.4 | 1.6 | N/R | N/R |
| AA417208 | ESTs | 1.4 | 1.9 | N/D | N/D |
| AI018181 | ESTs | 1.4 | 1.8 | N/R | N/R |
| N76371 | ESTs | 1.4 | 1.4 | N/D | N/D |
| AA167722 | ESTs | 1.4 | 1.4 | N/R | N/R |
| AI801966 | ESTs | 1.4 | 1.7 | N/R | N/R |
| AA436947 | ESTs | 1.4 | 1.6 | N/R | N/R |
| AF038201 | clone 23555 mRNA sequence | 1.4 | 1.4 | N/R | N/R |
| AA313794 | clone 25030 mRNA sequence | 1.4 | 1.5 | N/R | N/R |
| D87074 | KIAA0237 gene product | 1.4 | 1.6 | −1.3 | 1.3 |
| AB018268 | KIAA0725 gene product | 1.4 | 1.4 | N/R | N/R |
| AB006631 | KIAA0293 gene product | 1.4 | 1.4 | N/R | N/R |
| D82343 | neuroblastoma (nerve tissue) protein | 1.4 | 1.8 | −1 | 2.3 |
| L10333 | reticulon 1 | 1.4 | 1.4 | 1.4 | 1.3 |
| AI937119 | ESTs | 1.5 | 1.6 | N/R | N/R |
| AI831729 | ESTs | 1.5 | 1.4 | N/R | N/R |
| AA707689 | ESTs, Weakly similar to cAMP-regulated | 1.5 | 1.7 | N/R | N/R |
| | guanine nucleotide exchange factor II | | | | |
| AA424109 | ESTs, Weakly similar to gene pp21 protein | 1.5 | 1.4 | N/R | N/R |
| Z99387 | ESTs | 1.6 | 1.8 | N/R | N/R |
| AI004627 | ESTs | 1.6 | 2.4 | N/R | N/R |
| AI306588 | ESTs | 1.7 | 1.5 | N/D | N/D |
| AI827127 | ESTs, Highly similar to KIAA0195 | 1.8 | 1.4 | N/D | N/D |
| AA679059 | ESTs | 12.8 | 20.4 | N/R | N/R |
| AA912329 | ESTs | 14.9 | 20.4 | N/R | N/R |

TABLE 4
Genes which meet the criteria for differential expression on the oligonucleotide
(A-1, A-2) arrays: Data from cDNA (GS-1, GS-2) arrays have been added
were possible

| | | | | | |
|---|---|---|---|---|---|
| Accession | Gene Name | A-1 | A-2 | GS-1 | GS-2 |

Structural Proteins

| | | | | | |
|---|---|---|---|---|---|
| M26880 | ubiquitin C | −1.5 | −1.5 | N/R | N/R |
| M64571 | microtubule-associated protein 4 | −1.4 | −1.5 | −1.1 | −1 |

Proteases

| | | | | | |
|---|---|---|---|---|---|
| X04106 | calcium dependent protease (small subunit) | 1.4 | 1.5 | N/R | N/R |
| D38048 | proteasome subunit z | −1.7 | −1.4 | N/R | N/R |

Receptors, Channels and other Synaptic Proteins

| | | | | | |
|---|---|---|---|---|---|
| S67368 | gamma-aminobutyric acid (Gaba) A receptor | −1.7 | −1.4 | N/R | N/R |
| | beta 2 subunit | | | | |
| U01824 | glutamate/aspartate transporter II | −5.1 | −1.5 | 1.1 | −1.9 |

Cell-Cell or Cell-Matrix Interactions

| | | | | | |
|---|---|---|---|---|---|
| J04182 | lysosomal membrane glycoprotein-1 | −1.6 | −1.5 | N/D | N/D |
| | (LAMP1) | | | | |
| X52947 | cardiac gap junction protein | −1.4 | −1.8 | −1 | −2.1 |
| L46720 | autotaxin-t (atx-t) | −1.6 | −1.8 | N/R | N/R |

Immune or Inflammatory Response

| | | | | | |
|---|---|---|---|---|---|
| M37245 | Ig superfamily cytotoxic T-lymphocyte- | −1.6 | −1.4 | N/R | N/R |
| | associated protein (CTLA-4) | | | | |
| X00274 | HLA-DR alpha heavy chain a class II | −1.7 | −1.6 | N/R | N/R |
| | antigen of the major histocompatibility | | | | |
| | complex (MHC) | | | | |

Metabolism

| | | | | | |
|---|---|---|---|---|---|
| U91316 | Human acyl-CoA thioester hydrolase | 1.4 | 2.6 | 1.1 | −1.2 |
| X13238 | cytochrome c oxidase subunit VIc | 1.4 | 1.5 | N/R | N/R |
| X76228 | vacuolar H+ ATPase E subunit | 1.5 | 1.8 | N/R | N/R |
| X83218 | ATP synthase | 1.5 | 1.5 | N/R | N/R |
| M19045 | lysozyme mRNA | −1.6 | −2.5 | N/R | N/R |
| M98539 | prostaglandin D2 synthase | −2 | −2.2 | N/R | N/R |
| X01677 | liver mRNA for glyceraldehyde-3-phosphate | −1.9 | −1.6 | N/R | N/R |
| | dehydrogenase | | | | |
| Z35093 | SURF-1 | −1.4 | −1.7 | N/R | N/R |

Cell Signaling

| | | | | | |
|---|---|---|---|---|---|
| J04046 | calmodulin | 1.4 | 1.4 | N/R | N/R |
| M29551 | calcineurin A2 | 2.4 | 2.5 | 1.2 | 1.3 |
| U82532 | GDI-dissociation inhibitor RhoGDIgammma | 1.4 | 2.4 | N/R | N/R |
| X56468 | 14.3.3 protein, a protein kinase regulator | 1.7 | 1.4 | 1.1 | 1.1 |
| X73478 | hPTPA | 2.1 | 2.5 | 1.2 | 1.1 |
| Z15108 | protein kinase C zeta | 1.4 | 1.8 | 1.1 | 1 |
| D14889 | small GTP-binding protein, S10 | −2.2 | −1.5 | −1.2 | 1.6 |
| L77886 | protein tyrosine phosphatase | −1.5 | −1.4 | N/R | N/R |
| M34181 | testis-specific cAMP-dependent protein | −2 | −1.7 | 1.2 | 1.2 |
| | kinase catalytic subunit (C-beta isoform) | | | | |
| M74491 | ADP-ribosylation factor 3 | −1.5 | −1.4 | 1.2 | 1.1 |

Molecular Chaperones and other Carrier Proteins

| | | | | | |
|---|---|---|---|---|---|
| J04988 | 90 kD heat shock protein gene | 1.4 | 1.5 | N/R | N/R |
| L26336 | heat shock protein HSPA2 gene | -3 | −1.6 | N/R | N/R |
| M22382 | mitochondrial matrix protein P1 (nuclear | −1.8 | −1.8 | 1.4 | 1.2 |
| | encoded) | | | | |

Transcription or Protein Synthesis

| | | | | | |
|---|---|---|---|---|---|
| L19779 | histone H2A.2 | 1.4 | 1.5 | N/R | N/R |
| L34587 | RNA polymerase II elongation factor SIII, | 1.4 | 2.4 | N/R | N/R |
| | p15 subunit | | | | |
| M17885 | acidic ribosomal phosphoprotein P0 | −1.4 | −1.4 | N/R | N/R |
| U09587 | glycyl-tRNA synthetase | −1.5 | −2.1 | N/R | N/R |
| U14968 | ribosomal protein L27a | −2 | −2.2 | N/R | N/R |

Cell Cycle Regulation: Growth, Proliferation and Death

| | | | | | |
|---|---|---|---|---|---|
| U29953 | pigment epithelium-derived factor | 1.6 | 1.4 | 1.1 | 1.2 |
| M63379 | TRPM-2 protein | −1.7 | −1.5 | −1.1 | −1.3 |

Other

| | | | | | |
|---|---|---|---|---|---|
| K02215 | angiotensinogen | −1.7 | −1.6 | N/R | N/R |
| S95936 | transferrin | −3.1 | −1.9 | N/R | N/R |
| U20908 | melanoma ubiquitous mutated protein | −1.6 | −1.4 | N/R | N/R |
| | (MUM-1) | | | | |

Unknown

| | | | | | |
|---|---|---|---|---|---|
| D38522 | KIAA0080 gene product | −1.5 | −1.7 | 1.3 | 1 |
| D42043 | KIAA0084 gene product | −1.4 | −2.3 | N/R | N/R |
| D79996 | KIAA0174 gene product | −1.6 | −2 | −1.1 | −1.2 |

Expression of several genes involved in synaptic transmission are altered in the alcoholic samples. In particular, the expression of α-synuclein is increased (Table 3). α-synuclein is an activity-dependent regulator of dopaminergic neurotransmission (Abeliovich et al., 2000), a process central to drug dependence, and both α and β-synuclein may have a role in neurodegeneration (Galvin et al., 1999). Similarly, there is increased expression of the AMPA1 receptor subunit, RGS-4, and N-ethylmaleidmide-sensitive factor (Table 3) while an inwardly-rectifying potassium channel (Table 3), the gamma-aminobutyric acid A (GABA_{α}) receptor beta-2 subunit and ADP ribosylation factor-3 (Table 4) genes show decreases in expression in the alcoholics. Some inwardly rectifying potassium channels are likely in vivo targets of alcohol action (Lewohl et al., 1999) and are modulated by RGS proteins which also regulate the G-protein signaling family of proteins by acting as GTPase activating proteins to modulate hormone and neurotransmitter receptor mediated signaling (Hepler 1999). Importantly, studies in animals show that prolonged ethanol treatment can increase expression of the GluR1 glutamate receptor subunit (Ortiz et al., 1995). Although the number of genes studied in animal models of alcoholism are limited, the concordance with human alcoholism for GluR1 and GFAP suggests that expression changes in alcoholics are due to alcohol consumption rather than confounding factors such as nutrition or trauma.

A cluster of genes known to regulate cell proliferation is down-regulated in the alcoholic samples. This included the genes coding for requiem, p53-binding protein, a discoidin-domain receptor family member and two CDC-like kinases. p53-Binding protein interacts with wildtype p53, the anti-apoptotic gene Bcl-2, and the p65(RelA) subunit of NFκB. (Yang J P, Hori M, Takahashi N, Kawabe T, Kato H, Okamoto T (1999) NF-kappaB subunit p65 binds to 53BP2 and inhibits cell death induced by 53BP2. Oncogene 18:5177-86). p53-Binding protein enhances p53-mediated transcriptional activation and may play a central role in the regulation of apoptosis and cell growth. Discoidin domian receptor 1, a member of the receptor tyrosine kinase family (Sakuma et al., 1996) is known to be regulated by p53. This tyrosine kinase is activated by binding of collagen and is thought to mediate cellular responses to the extracellular matrix (Vogel 1999). Two other genes, Nel-like 2 and RAN, also known to function in regulation of cell growth, are up-regulated in the alcoholics. Some of these responses, such as the down-regulation of requiem and p53-binding protein, suggest an “anti-apoptotic” response. This could either be directly due to ethanol or be a compensatory response to ethanol acting in a pro-apoptotic manner. In this regard, alcohol is shown to have pro-apoptotic effects in both developing and adult animals (Ikonomidou et al., 2000; Wu and Cederbaum 2000).

Because alcoholics often die at a relatively young age, one difficulty using autopsy tissue is matching of age. In case group 1, the alcoholics are an average of 10 years younger than controls (not significantly different, t-test), and in case group 2 they are 16 years younger than controls (p=0.03, t-test). Thus, it is important to consider if age may contribute to the differences in gene expression seen between controls and alcoholics. The first array study of changes in brain gene expression in aging documents increased expression of 70 genes and decreased expression of 86 genes (using a >1.4 fold criterion and a 6347 gene array) (Lee et al., 2000 and supplementary material). This comparison of neocortex of 5 and 30 month old mice does not detect changes in most of the genes that were found were altered between alcoholics and controls. For example, the cluster of myelin gene changes that is so prominent in this study does not appear in the aging study. However, expression of apolipoprotein D, alpha-synuclein, macrophage colony stimulating factor 1, myelin oligodendrocyte glycoprotein and GFAP, changes in both studies and the direction of change is such that age differences could contribute to the changes in gene expression seen in alcoholics. The limited overlap between the changes in gene expression in alcoholism and aging suggests that differences in age in the subjects did not contribute to most of the changes observed.

Gene Expression Changes, Prognostic and Diagnostic Medical Information

The present invention demonstrates that it is possible to identify the presence, absence, or modifications in the expression of genes related to alcoholism in the blood of patients. By using blood as the source of the nucleic acids for detection, the present invention greatly simplifies and makes detection of patients or potential patients more amenable to widespread use. One or more alcoholism-specific nucleic acids attach to the surface of the substrate under conditions apparent to those of skill in the art of molecular biology.

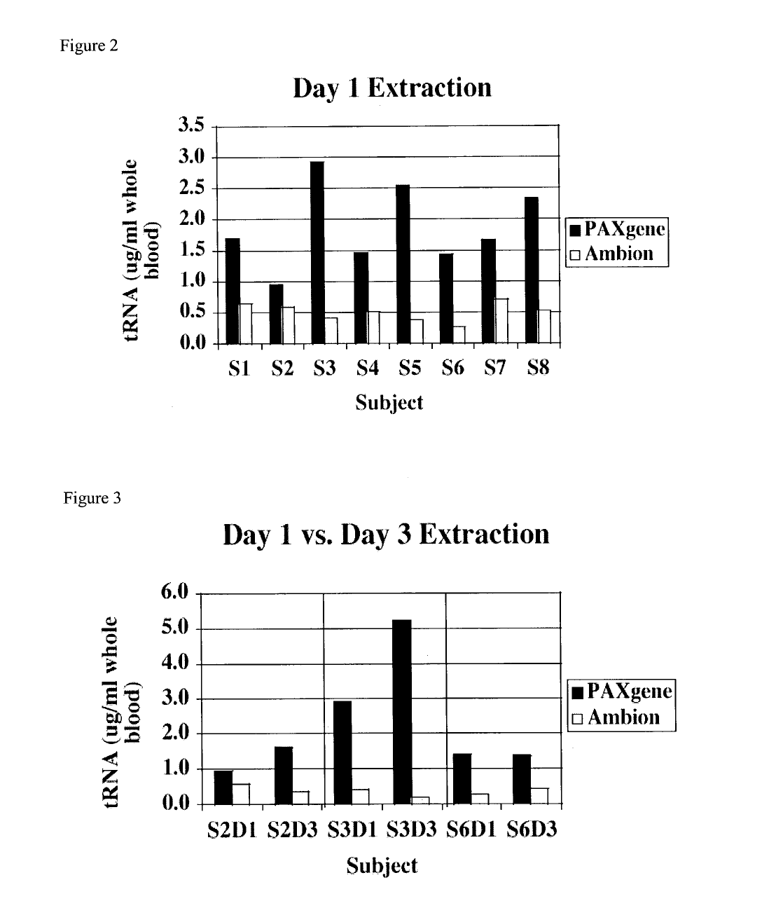

FIG. 2 is a graph with the results from blood tRNA after one day using the PAX gene and Ambion systems as probes to evalute the level of expression detectable in blood from subjects for genes associated with alcoholism. Alternatively, other probes may be used to compare gene expression. Based on the predominant genes identified in blood, which match the genes identified from brain tissue, the presence, absence or modification in the expression of genes associated with alcoholism may be detected by taking a blood sample and testing the nucleic acids extracted from the sample against an alcohol specific array of the present invention. Appropriate controls are included in the array. Using the present invention even a single predominant gene may be used to test for alcoholism, however, more than one gene may be used to increase the accuracy of the results. A single gene assay is useful for preliminary screening, e.g., for use by law enforcement agencies to pre-screen inmates so that specific resources may be focused on that patient to aid in their treatment. In this manner, the patient is identified early in the cycle and those patients receive the treatment that they need, without expending resources on patients that are not at risk.

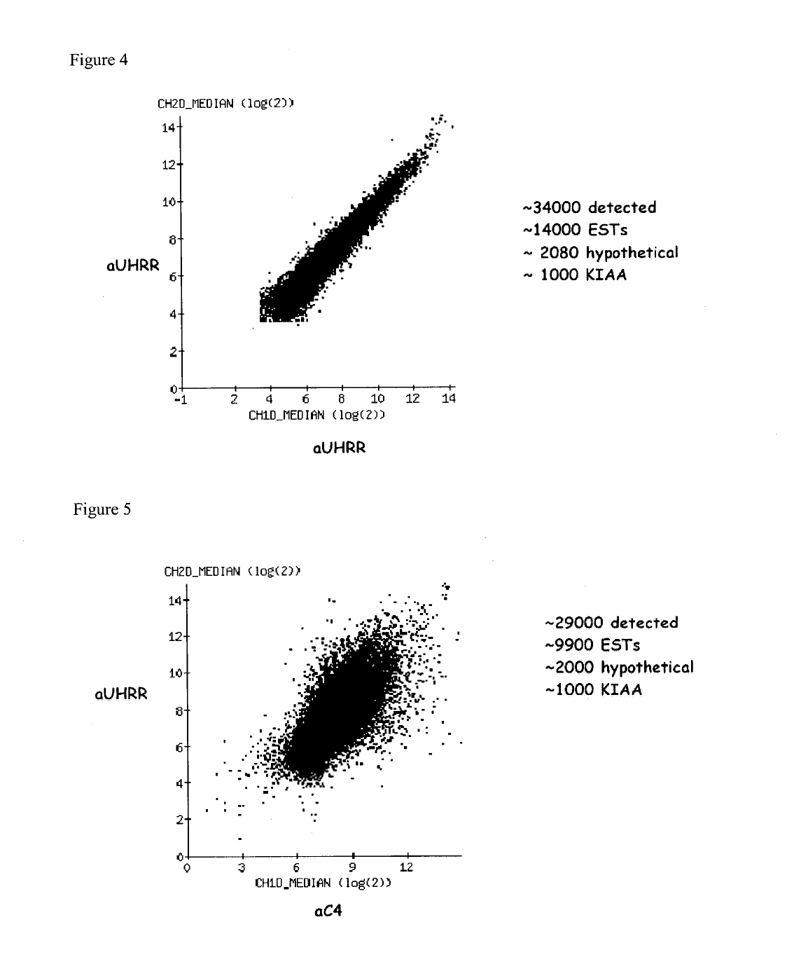

FIG. 3 is a graph comparing the levels of genes from different subjects at day 1 and day 3. FIGS. 4 and 5 are reference graph and a graph comparing reference to human blood sample gene expression detected from an individual blood sample. The underlying data that was used to obtain FIGS. 2-5, follows. The underlying data demonstrates that a number of the same genes detected in blood that have been identified in previous studies of human brain, outlined herein above, and therefore the genes identified from alcoholic brain samples correlate and are detectable in blood.

Examples of “alcoholism-specific nucleic acids” for detection from a blood sample include at least a portion of the following genes: apolipoprotein (various); aquaporin (various); CD44 antigen (homing function and Indian blood group system); dopamine receptor D2; histamine receptor H1; Homo sapiens beta-1 adrenergic receptor mRNA, 3′ UTR; myelin associated glycoprotein; myelin basic protein; myelin gene expression factor 2; myelin oligodendrocyte glycoprotein; myelin protein zero-like 1; myelin transcription factor 1-like; myelin-associated oligodendrocyte basic protein; neuronal pentraxin I; neuronal pentraxin Ii; neuronal pentraxin receptor; neuronal potassium channel alpha subunit; neuronal protein; neuronal protein 17.3; neuronal Shc; neuronal Shc adaptor homolog; neuronal specific transcription factor DAT 1; neuronatin; neuron-specific protein; neuronal pentraxin receptor; neuropeptide Y receptor Y1; neurotensin receptor 2; neurotrophic tyrosine kinase, receptor, type 2; peripheral myelin protein 2; peripheral myelin protein 22; phospholipase A2 receptors; proteolipid protein 2; sodium channel, voltage gated, type VIII, alpha polypeptide; sodium channel, voltage-gated, type II, beta polypeptide; syntaxin (various); various transporters.

The present invention can provide a method for the detection, monitoring, and diagnosis of alcoholism and alcohol-related diseases. The lack of standardized methods to determine the onset or scope of alcoholism, in addition to inadequate measures that do not monitor disease progression have contributed to the need to search for alternatives to existing detection, monitoring, and diagnostic methods. The present invention is also suggestive of an extensive, but selective, re-programming of myelin gene expression. The coordinate regulation of multiple myelin genes suggests a possible toxic action of ethanol on oligodendrocyte function or survival. The toxic action may provide a molecular basis for the susceptibility of alcoholics to white-matter loss and demyelinating diseases. In addition, the unanticipated changes in novel neuronal genes such as synuclein and in cell cycle gene expression provide new opportunities for understanding, and perhaps halting or reversing, the changes in brain structure and function that are hallmarks of alcoholism.

1. A device for detecting the presence of genes related to alcoholism comprising:
a substrate; and
one or more alcoholism-specific nucleic acids attached to the substrate.

2. A device of claim 1 wherein the substrate comprises a microfabricated solid surface to which molecules may be attached through either covalent or non-covalent bonds.

3. The device of claim 2 wherein the substrate further comprises Langmuir-Bodgett films, glass, functionalized glass, germanium, silicon, PTFE, polystyrene, gallium arsenide, gold, silver, or any materials comprising amino, carboxyl, thiol or hydroxyl functional groups incorporated on a planar or spherical surface.

4. The device of claim 1 wherein one or more alcoholism-specific nucleic acids comprise human nucleic acid target elements of one or more peptide nucleic acids with different determinable sequences.

5. The device of claim 4 wherein the human nucleic acid target elements comprise one or more peptides, each at a different locations on the substrate at a density of 100 to 10,000 target elements per square centimeter.

6. The device of claim 4 wherein the human nucleic acid target elements comprise genomic DNA, cDNA, oligonucleotides, RNA, single-stranded or double-stranded or any chemical modifications thereof.

7. The device of claim 4 wherein the human nucleic acid target elements are portions of alcoholism-specific genes with sequences specific to structural, metabolic, transcriptional or other genes for cell signaling, immune response, and or cell-cell interactions that are expressed by alcoholics or alcohol abusers, including M6 neuronal glycoprotein, myelin associated glycoprotein, myelin-associated oligodendrocyte basic protein, myelin basic protein, myelin proteolipid protein, myelin-oligodendrocyte glycoprotein, myelin protein Po, oligodendrocyte-myelin glycoprotein, PMP2, PMP22, MAL gene, ApoD, ApoE, carbonic anhydrase II, 2′,3′-cyclic nucleotide 3′-phosphodiesterase, Galactocerebrosidase, Transaldolase, UDP-galactose ceramide galactosyltransferase, MyT 1, Puralpha, Edg-2, glial fibrillary acidic protein, keratin 6B, beta III spectrin, protease, serine, 9 (neurosin), proprotein convertase subtilisin/kexin type 4, calpain, large polypeptide L3, protease, serine, 11 (IGF binding), transmembrane protease, serine 2, endothelin receptor type B-like (GPCR 37), aquaporin 1 (channel-forming integral protein), potassium inwardly-rectifying channel, subfamily J, member 10, glutamate receptor, AMPA 1, N-ethylmaleimide-sensitive factor, EGF-containing fibulin-like extracellular matrix protein 1, CD44 antigen, cadherin 18, tetraspan NET-6, interferon, gamma-inducible protein 16, major histocompatibility complex, class II, DR beta 1, small inducible cytokine subfamily C, member 1, epoxide hydrolase 1, microsomal (xenobiotic), proline dehydrogenase (proline oxidase), glutathione S-transferase M5, ubiquinol-cytochrome c reductase core protein II, serine/threonine kinase, TU3A protein (also known as dominant rapamycin resistance 1, DRR1), secreted frizzled-related protein 1, GTP binding protein, phospholipase A2 (14-3-3 protein), platelet-activating factor acetylhydrolase, isoform Ib, alpha subunit, regulator of G-protein signaling 4 (RGS-4), synuclein, alpha, ribophorin II, 130 kD Golgi-localized phosphoprotein, autoimmune regulator (automimmune polyendocrinopathy candidiasis ectodermal dystrophy), SRY-box 9 transcription factor, spliceosome-associated protein (U2 snRNP), retinoid X receptor, gamma, nuclear transcription factor, X-box binding 1, TATA box binding protein-associated factor, RNA polymerase II, F, basic transcription factor 3, macrophage stimulating 1 (hepatocyte growth factor-like), LIM domain only 2 (rhombotin-like 1), bone morphogenetic protein 7 (osteogenic protein 1), requiem, apoptosis response zinc finger gene, discoidin domain receptor family, member 1, CDC-like kinase 2, CDC-like kinase 1, tumor protein p53-binding protein, 2, Human growth/differentiation factor 1, RACH1 (complements rad1-1 cell cycle checkpoint mutant), RAN (member RAS oncogene family), nel-like 2, sarcolipin, KIAA0043 gene product, ESTs, cysteine and glycine-rich protein 1, conserved gene amplified in osteosarcoma, KIAA0027 gene product, Homo sapiens clone 23916, KIAA0202 gene product, selenoprotein P, plasma, 1, chromosome 16 BAC clone CIT987SK-A-69G12, upregulated by 1,25-dihydroxyvitamin D-3, tight junction protein 2, HREV107-like protein, clone 23555 mRNA sequence, clone 25030 mRNA sequence, KIAA0237 gene product, KIAA0725 gene product, KIAA0293 gene product, neuroblastoma (nerve tissue) protein, reticulon 1, ESTs weakly similar to cAMP-regulated guanine nucleotide exchange factor II, ESTs weakly similar to gene pp21protein, or ESTs highly similar to KIAA0195, ubiquitin C, microtubule-associated protein 4, calcium dependent protease (small subunit, proteasome subunit z, gamma-aminobutyric acid (GABA) A receptor beta 2 subunit, glutamate/aspartate transporter II, lysosomal membrane glycoprotein-1 (LAMP 1), cardiac gap junction protein, autotaxin-t (atx-t), Ig superfamily cytotoxic T-lymphocyte-associated protein (CTLA-4), HLA-DR alpha heavy chain a class II antigen of the major histocompatibility complex (MHC), Human acyl-CoA thioester hydrolase, cytochrome c oxidase subunit Vic, vacuolar H+ATPase E subunit, ATP synthase, lysozyme mRNA, prostaglandin D2 synthase, liver mRNA for glyceraldehyde-3-phosphate dehydrogenase, SURF-1, calmodulin, calcineurin A2, GDI-dissociation inhibitor RhoGDlgammma, 14.3.3 protein (a protein kinase regulator), hPTPA, protein kinase C zeta, small GTP-binding protein, S10, protein tyrosine phosphatase, testis-specific cAMP-dependent protein kinase catalytic subunit (C-beta isoform), ADP-ribosylation factor 3, 90 kD heat shock protein gene, heat shock protein HSPA2 gene, mitochondrial matrix protein P1 (nuclear encoded), histone H2A.2, RNA polymerase II elongation factor SIII p15 subunit, acidic ribosomal phosphoprotein P0, glycyl-tRNA synthetase, ribosomal protein L27a, pigment epithelium-derived factor, TRPM-2 protein, angiotensinogen, transferrin, melanoma ubiquitous mutated protein (MUM-1), KIAA0080 gene product, KIAA0084 gene product, or a KIAA0174 gene product.

8. The device of claim 1 wherein the alcoholism-specific nucleic acids come in contact with a sample.

9. The device of claim 8 wherein the sample comprises macromolecules, in whole or in part, including blood plasma, urine, semen, saliva, lymph fluid, meningeal fluid, amniotic fluid, glandular fluid, and cerebrospinal fluid, cells, or any other fluid, cell or body tissue preparation.

10. The device of claim 8 wherein the sample is collected from a person who is considered to be alcoholic, alcohol abusive or have an alcohol-related disease.

11. The device of claim 8 wherein portions of the sample bind specifically to one or more human nucleic acid target elements.

12. The device of claim 8 wherein the binding is detected by a light source, capacitor, ion or plasma beam, including light microscopy, radiography, chemiluminescence, fluorescence microscopy, confocal microscopy, interferometry, surface plasma resonance, mass spectroscopy, atomic force microscopy, scanning tunneling microscopy.

13. The device of claim 8 wherein the alcoholism-specific nucleic acid and the sample come in contact under selective binding conditions.

14. The device of claim 13 wherein the selective binding conditions provide information that can be collected as a detectable change in signal intensity.

15. The device of claim 14 wherein the information is recorded by a computer.

16. The device of claim 15 wherein the computer records the information, stores the information in a database, and or displays the information.

17. The device of claim 14 wherein the information includes the location and magnitude of the detectable change at each human nucleic acid target element.

18. The device of claim 15 wherein an information ratio is determined between the sample information and a control information.

19. The device of claim 15 wherein the control information is obtained from a sample collected from a person who is not an alcoholic or alcohol abusive, and or from a person who does not have an alcohol-related disease, under like conditions to that of the sample.

20. The device of claim 18 wherein the information yields gene expression information and diagnostic and or prognostic medical information about the person.

21. A method for identifying alcoholism-specific genes, comprising the steps of:
contacting a sample obtained from a person considered to be alcoholic or alcohol abusive or has an alcohol-related disease with a substrate comprising one or more genes associated with alcoholism; and
comparing the level of expression of the sample with that of a non-alcoholic control sample, wherein changes in expression level are correlated with alcoholism.

22. The method of claim 21 wherein the sample comprises macromolecules, in whole or in part, including blood plasma, urine, semen, saliva, lymph fluid, meningeal fluid, amniotic fluid, glandular fluid, and cerebrospinal fluid, cells, or any other fluid, cell or body tissue preparation.

23. The method of claim 21 wherein the substrate comprises a microfabricated solid surface to which molecules may be attached through either covalent or non-covalent bonds.

24. The method of claim 21 wherein the substrate further comprises Langmuir-Bodgett films, glass, functionalized glass, germanium, silicon, PTFE, polystyrene, gallium arsenide, gold, silver, or materials comprising amino, carboxyl, thiol or hydroxyl functional groups incorporated on a planar or spherical surface.

25. The method of claim 21 wherein human nucleic acid target elements are attached to the substrate.

26. The method of claim 25 wherein the human nucleic acid target elements comprise genomic DNA, cDNA, oligonucleotides, RNA, single-stranded or double-stranded and any chemical modifications thereof.

27. The method of claim 25 wherein the human nucleic acid target elements further comprise determinable sequences from alcoholism-related genes.

28. The method of claim 25 wherein each human nucleic acid target elements contains at least one peptide at a different location on the substrate at a density of 100 to 10,000 target elements per square centimeter.

29. The method of claim 25 wherein the human nucleic acid target elements are portions of structural, metabolic, transcriptional or other genes, including ones that code for proteases, receptors, channels, synaptic proteins, cell-cell or cell-matrix interactions, immune or inflammatory responses, cell signaling, molecular chaperones or other carrier proteins, molecular synthesis, cell cycle regulation, cell growth, cell proliferation, or cell death.

30. The method of claim 21 wherein the substrate is a microarray.

31. The method of claim 21 wherein the binding includes binding of portions of the sample specifically to one or more human nucleic acid target elements.

32. The method of claim 21 wherein the binding occurs under selective binding conditions.

33. The method of claim 21 wherein the binding is detected by a light sources, capacitor, ion or plasma beam, including light microscopy, radiography, chemiluminescence, fluorescence microscopy, confocal microscopy, interferometry, surface plasma resonance, mass spectroscopy, atomic force microscopy, scanning tunneling microscopy.

34. The method of claim 21 wherein the binding provides information that can be collected as a detectable change in signal intensity.

35. The method of claim 34 wherein the information is recorded by a computer.

36. The method of claim 35 wherein the computer records the information, stores the information in a database, and or displays the information.

37. The method of claim 34 wherein the information includes the location and magnitude of the detectable change at each human nucleic acid target element.

38. The method of claim 34 wherein an information ratio is determined between the sample information and a control information.

39. The method of claim 38 wherein the control information is obtained from a sample collected from a person who is not an alcoholic or alcohol abusive, and or from a person who does not have an alcohol-related disease, under like conditions to that of the sample.

40. The method of claim 38 wherein the information yields gene expression information and diagnostic and or prognostic medical information about the person.

41. A device for detecting the presence of genes related to alcoholism from a blood sample comprising a substrate comprising one or more alcoholism-specific nucleic acids and one or more control nucleic acids, wherein the expression of one or more alcoholism-specific nucleic acids is detected from the nucleic acids collected from a sample of blood.

42. The device of claim 41, wherein the human nucleic acid target elements comprise genomic DNA, cDNA, oligonucleotides, RNA, single-stranded or double-stranded or any chemical modifications thereof.

43. The device of claim 41, wherein the nucleic acid of the blood sample comprise a portion of alcoholism-specific genes selected from the group consisting of apolipoprotein; aquaporin; CD44 antigen; dopamine receptor D2; histamine receptor H1; Homo sapiens beta-1 adrenergic receptor mRNA, 3′ UTR; myelin associated glycoprotein; myelin basic protein; myelin gene expression factor 2; myelin oligodendrocyte glycoprotein; myelin protein zero-like 1; myelin transcription factor 1-like; myelin-associated oligodendrocyte basic protein; neuronal pentraxin I; neuronal pentraxin Ii; neuronal pentraxin receptor; neuronal potassium channel alpha subunit; neuronal protein; neuronal protein 17.3; neuronal Shc; neuronal Shc adaptor homolog; neuronal specific transcription factor DAT1; neuronatin; neuron-specific protein; neuronal pentraxin receptor; neuropeptide Y receptor Y1; neurotensin receptor 2; neurotrophic tyrosine kinase, receptor, type 2; peripheral myelin protein 2; peripheral myelin protein 22; phospholipase A2 receptors; proteolipid protein 2; sodium channel, voltage gated, type VIII, alpha polypeptide; sodium channel, voltage-gated, type II, beta polypeptide; and syntaxin, and combinations thereof.

44. A method for screening for one or more alcoholism-specific genes, comprising the steps of:
contacting nucleic acids obtained from a blood sample with a substrate comprising one or more genes associated with alcoholism; and
comparing the level of expression of the sample with that of a non-alcoholic control sample, wherein changes in expression level are correlated with alcoholism.

45. The method of claim 44, wherein the human nucleic acid target elements comprise genomic DNA, cDNA, oligonucleotides, RNA, single-stranded or double-stranded or any chemical modifications thereof.

46. The device of claim 44, wherein the nucleic acid of the blood sample comprise a portion of alcoholism-specific genes selected from the group consisting of apolipoprotein; aquaporin; CD44 antigen; dopamine receptor D2; histamine receptor H1; Homo sapiens beta-1 adrenergic receptor mRNA, 3′ UTR; myelin associated glycoprotein; myelin basic protein; myelin gene expression factor 2; myelin oligodendrocyte glycoprotein; myelin protein zero-like 1; myelin transcription factor 1-like; myelin-associated oligodendrocyte basic protein; neuronal pentraxin I; neuronal pentraxin Ii; neuronal pentraxin receptor; neuronal potassium channel alpha subunit; neuronal protein; neuronal protein 17.3; neuronal Shc; neuronal Shc adaptor homolog; neuronal specific transcription factor DAT1; neuronatin; neuron-specific protein; neuronal pentraxin receptor; neuropeptide Y receptor Y1; neurotensin receptor 2; neurotrophic tyrosine kinase, receptor, type 2; peripheral myelin protein 2; peripheral myelin protein 22; phospholipase A2 receptors; proteolipid protein 2; sodium channel, voltage gated, type VIII, alpha polypeptide; sodium channel, voltage-gated, type II, beta polypeptide; and syntaxin, and combinations thereof.
